(19) United States
(12) Patent Application Publication
(10) Pub. No.: US 2015/0147409 A1
(43) Pub. Date: May 28, 2015

(54) ADIPOSE STROMAL VASCULAR FRACTION-CONDITIONED MEDIUM

(71) Applicant: Indiana University Research and Technology Corporation, Indianapolis, IN (US)

(72) Inventors: Keith L. March, Carmel, IN (US); Brian H. Johnstone, Indianapolis, IN (US)

(21) Appl. No.: 14/550,400

(22) Filed: Nov. 21, 2014

Related U.S. Application Data
(60) Provisional application No. 61/907,642, filed on Nov. 22, 2013.

(51) Int. Cl. A61K 35/12; C12N 5/077

(52) U.S. Cl. CPC A61K 35/35; C12N 5/0653; C12N 2502/1305
USPC 424/574; 435/378

(57) ABSTRACT

A combination of therapeutic factors derived from non-adherent or poorly adherent stromal vascular fraction (SVF) cells exposed to protein-free basal medium are disclosed.

### CROSS REFERENCE TO RELATED APPLICATIONS

This application claims priority to U.S. Provisional Patent Application No. 61/907,642 filed on Nov. 22, 2013, which is hereby expressly incorporated by reference in its entirety.

### STATEMENT OF GOVERNMENT SUPPORT

This invention was made with government support under grant number T32 awarded by the National Institutes of Health. The U.S. Government has certain rights in the invention.

### BACKGROUND

The present disclosure relates generally to conditioned medium obtained from adipose tissue. In particular, the conditioned medium can be obtained from a non-adipocyte, stromal-vascular fraction.

Cell-based therapies, in particular stem cell-based therapies including human adipose-derived stem cells (ASC), have been shown to improve functional outcomes in cardiovascular and numerous other disease models, predominantly through secretion of beneficial factors that support the body's intrinsic repair and survival mechanisms. The lasting damage caused by stroke, for example, is limited by supplying these factors to protect brain cells from dying and stimulate replacement of those already lost.

Although cell-based therapies have shown great promise in regenerative medicine, there remains the theoretical risk that stem cells will form tumors or form emboli in blood vessels. In addition it is not possible to predict the effect of the in vivo environment on stem cell activity. Thus, undesirable alterations in stem cell function may occur after the cells are delivered to the patient. An additional problem with stem cell therapies relates to demonstrating that the potency and desired cell phenotype is maintained during production; especially, at large scales required for commercial production of cells.

One solution to remove these risks is to isolate the factors produced by stem cells in culture that are present in the culture medium (“conditioned medium”) for delivery after removing all cells from the final product. This results in a “cocktail” of factors that possesses the therapeutic benefits without the risk associated with stem cells. It is possible to characterize the factors present in conditioned medium, which will not change after delivery to the patient.

As provided herein, the present disclosure provides alternative conditioned medium. Particularly, the conditioned medium can be obtained from a non-adipocyte, stromal-vascular fraction.

### BRIEF DESCRIPTION

In one aspect, the present disclosure is directed to a conditioned medium obtained from digested adipose tissue.

In another aspect, the present disclosure is directed to a conditioned medium obtained from a non-adipocyte, stromal-vascular fraction.

In another aspect, the present disclosure is directed to a method of producing conditioned medium, the method comprising: digesting adipose tissue by contacting the adipose tissue with an enzyme; fractionating the adipose tissue to obtain a fractionated adipose tissue that comprises at least a stromal-vascular fraction; culturing cells obtained from the fractionated tissue in a culture medium; and separating the cells from the culture medium to produce the conditioned medium.

### BRIEF DESCRIPTION OF THE DRAWINGS

The disclosure will be better understood, and features, aspects and advantages other than those set forth above will become apparent when consideration is given to the following detailed description thereof Such detailed description makes reference to the following drawings, wherein:

FIG. 1 is a schematic illustrating isolation of stromal vascular fraction (SVF).

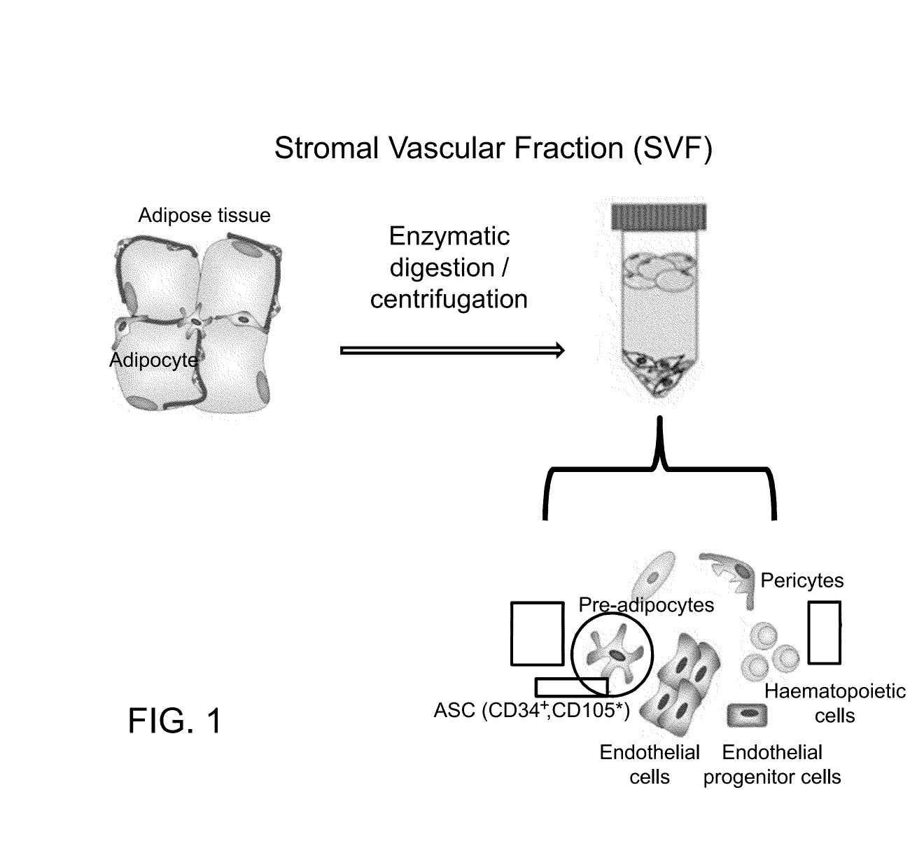

FIGS. 2A-2C show ASC-CM protection of neonatal rat brains from hypoxia-ischemia injury.

FIGS. 3A-3H demonstrate different secretion profiles of ASC-CM and SVF-CM.

FIG. 4 illustrates one exemplary method of the present disclosure for producing the conditioned medium.

FIG. 5 is a table summarizing SVF characteristics and briefly plated CM-producing SVF cells.

FIGS. 6A-6E show scatter plots of SVF cells and attached cells.

FIGS. 7A & 7B show scatter plots of SVF cells and attached cells.

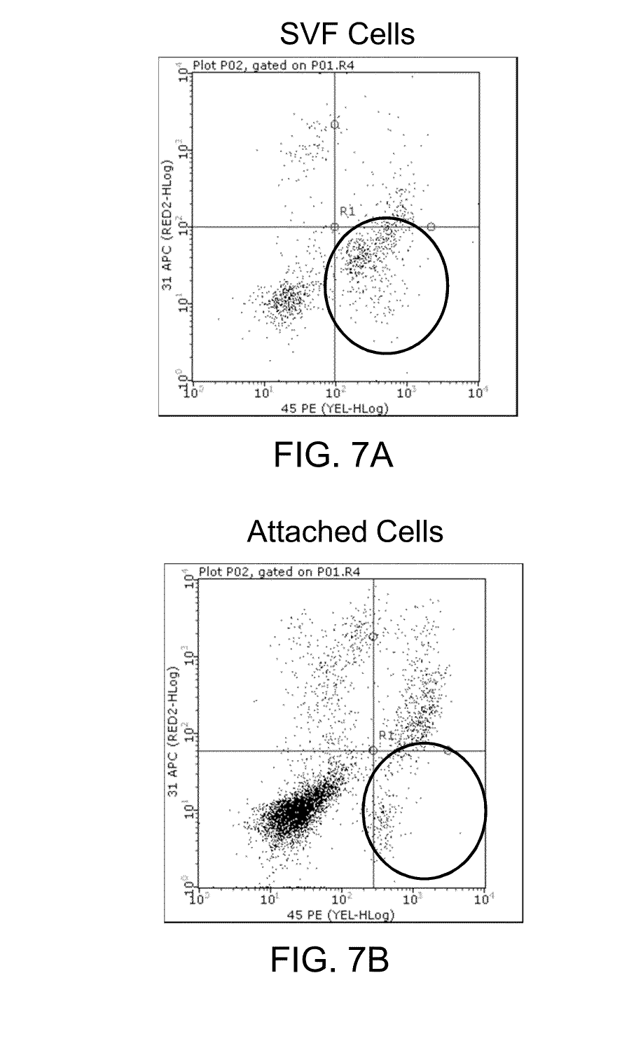

FIG. 8 is a graph illustrating vascular endothelial growth factor (VEGF) concentration in SVF-CM versus ASC-CM.

FIGS. 9A-9D are photomicrographs of cells at day 0 in culture, cells treated with basal medium, overnight plated (O/N plated) SVF, and non-adherent SVF-CM.

FIG. 10 is a graph illustrating that non-adherent SVF cell condition medium promotes endothelial cell proliferation.

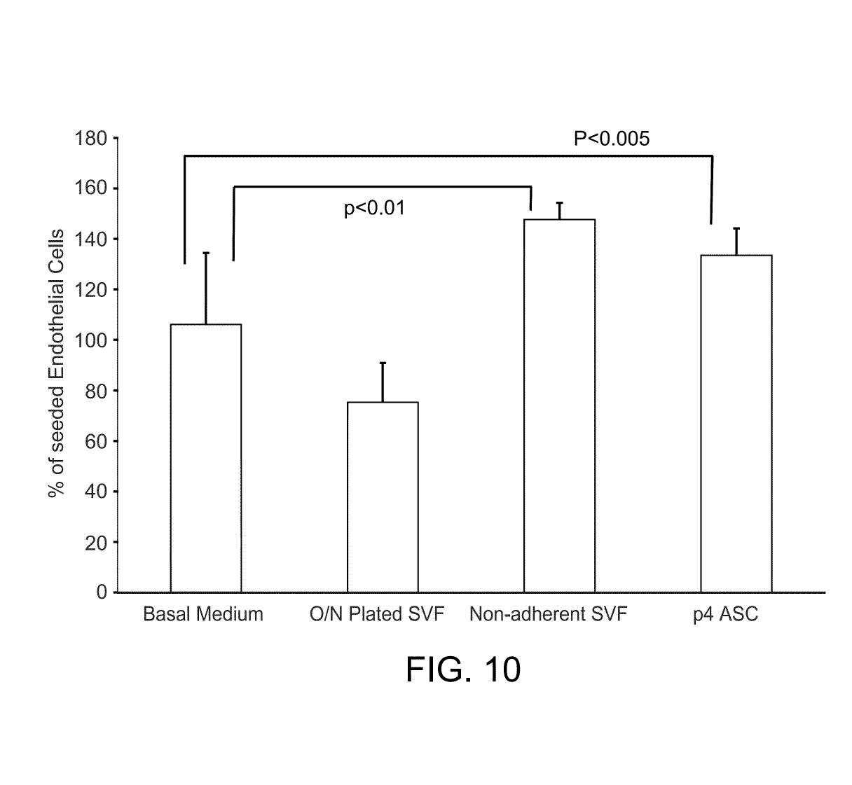

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred materials and methods are described below.

ASC are mesenchymal stem cells isolated from stromal-vascular fraction (SVF) obtained from enzymatically digested adipose tissue. The SVF also contains leukocytes and endothelial cells. ASC are commonly enriched based on their ability to selectively adhere within 24 hours of plating onto uncoated tissue culture plastic in rich media. The non-adhering cells, predominantly comprised of leukocytes and endothelial cells, are removed by aspiration and washing with buffer. The adherent ASC can then be expanded in rich medium until the desired passage and cell number are obtained, following which they can be subjected to a basal medium. The conditioned basal medium can then be collected.

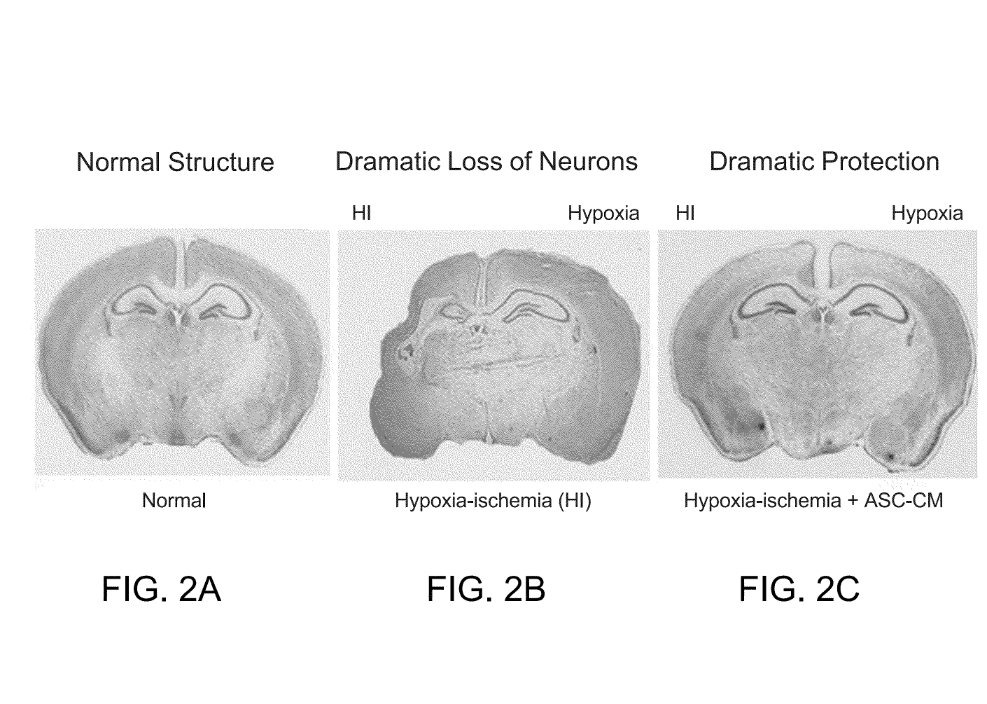

The conditioned medium (CM) derived from culturing and expanding ASCs, termed ASC-CM, has been shown to protect and repair damage in brains of rodents when delivered up to 36 hours after blocking blood flow to half of their brains (similar to a massive stroke) (FIGS. 2A-2C).

In accordance with the present disclosure, conditioned medium obtained from adipose tissue and methods of preparing are described. Conditioned medium obtained from freshly isolated or non-adherent population of SVF (stromal-vascular fraction) produces conditioned medium with biological activity that is at least as potent as adipose-derived stem cells conditioned medium (ASC-CM). Previously, it was generally assumed that the potency of factors obtained from SVF-derived cells was predominantly, if not wholly, contained within the stromal fraction of which ASC are the predominant cell type. Production of CM from the recently isolated or non-adhering fraction improves upon the current state of art by allowing for culturing in suspension culture, which simplifies the process and provides advantages related to obtaining higher yields of factors more quickly, including potentially when required (in an autologous paradigm) for a patient without the need for extended culture.

In one embodiment, conditioned medium is obtained from a freshly isolated population of SVF (stromal-vascular fraction) or a non-adherent population of SVF to produce conditioned medium with biological activity. In another embodiment, conditioned medium is obtained from adipose-derived stem cells to produce conditioned medium with biological activity.

The neuroprotective mechanism of action of ASC-CM has been explored in studies involving several types of neurons isolated from various regions of the brain, as well as using neural progenitors. ASC-CM protects against excitatory damage, oxidant stress, and generalized neuronal and hypoxic death in neuronal culture (see FIGS. 2A-2C) by the combined and synergistic action of several factors, including vascular endothelial growth factor (VEGF), IGF-1 (Insulin-like growth factor-1), NGF (Nerve growth factor), BDNF (Brain-derived neurotrophic factor), and HGF (Hepatocyte growth factor). ASC-CM also exhibits potent complementary activities to promote progenitor activity, including neuritogenesis (production of neurons).

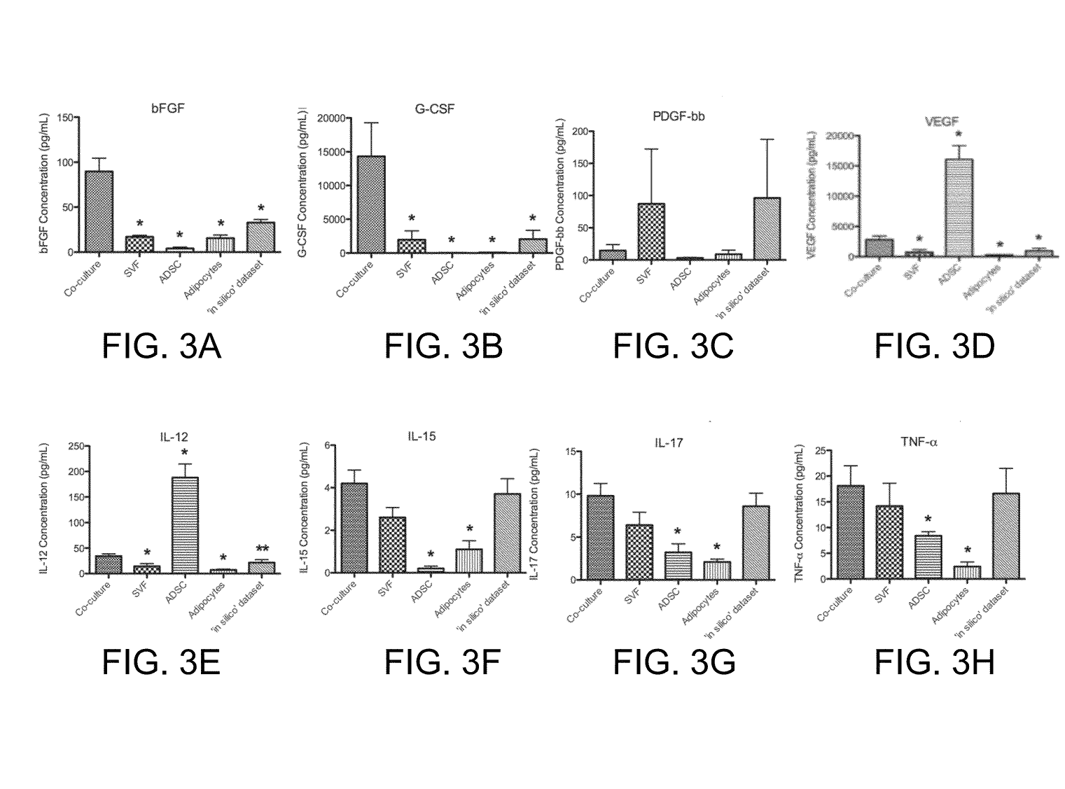

CM derived from non-adherent SVF cells (SVF-CM) possesses bioactivity such as, for example, the induction of endothelial cell proliferation. Non-adherent SVF cells can include, for example, leukocytes and endothelial cells. Further, SVF-CM possesses substantial amounts of VEGF, which can contribute to the effects on endothelial cells. Secretion profiles from ASC-CM and SVF-CM are shown in FIGS. 3A-3H.

In another aspect, the present disclosure is directed to a method of producing conditioned medium. The method includes: digesting adipose tissue by contacting the adipose tissue with an enzyme; fractionating the adipose tissue to obtain a fractionated adipose tissue that comprises at least a stromal-vascular fraction; culturing cells obtained from the fractionated tissue in a culture medium; and separating the cells from the culture medium to produce the conditioned medium.

Suitable enzymes for digesting adipose tissue are well known to those skilled in the art. Suitable enzymes can be, for example, trypsin, chymotrypsin, dispase, collagenase, hyaluronidase, papain, elastase, deoxyribonuclease 1, and pepsin A.

Suitable methods for fractionating the adipose tissue after being digested with an enzyme can be, for example, culturing the digested adipose tissue.

A particularly suitable method for fractionating the digested adipose tissue is by culturing the digested adipose tissue. After washing the digested cells to remove the enzyme used for digestion, the cells can be plated on a culture dish such as a plastic culture dish, for a time that is sufficient for a plurality of cells to settle out of the medium and contact the dish. The cells can then adhere to the culture dish, whereas other cells remain unattached to the dish. After culturing the cells for a sufficient amount of time, the unattached cells can be removed from the dish by pouring the culture medium that contains the unattached cells off of the dish, aspirating the culture medium that contains the unattached cells from the dish using a pipette and other methods for removing solutions. Cells that are adhered to the culture dish can be discarded or continued to be cultured by adding fresh culture medium as known to those skilled in the art. The cells contained within the culture medium that is removed represent the non-adherent cells. As with the attached cells, the non-adherent cells can be discarded or continued to be cultured by adding fresh culture medium as known to those skilled in the art. The non-adherent cells can suitably be cultured in suspension as known by those skilled in the art.

As known by those skilled in the art, cultured cells secrete various molecules into the medium in which they are cultured (referred to herein as “conditioned medium” or “CM”). The conditioned medium from cultures of attached cells and suspension cultures of non-adherent cells can be obtained using methods known by those skilled in the art. As described previously, the conditioned medium can be obtained from attached cells by pouring or aspirating. The conditioned medium can be obtained from suspension cultures by centrifuging the suspension culture to cause the cells of the suspension culture to pellet and collecting the condition medium using methods known to those skilled in the art.

The conditioned medium can be subjected to additional methods such as, for example, high-speed centrifugation, chromatography, dialysis, and other methods known to those skilled in the art to isolate and identify components contained within the conditioned medium.

### EXAMPLES

### Example 1

In this Example, conditioned medium obtained from freshly isolated non-adherent SVF (SVF-CM) was prepared and compared to ASC-CM.

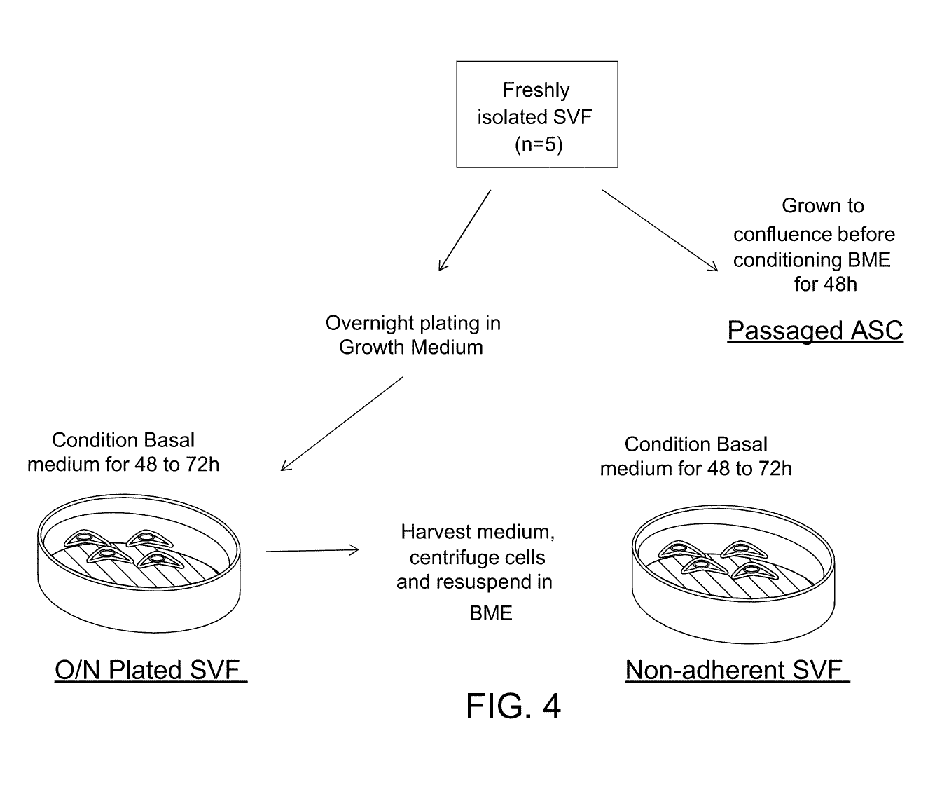

To prepare the SVF-CM, freshly isolated SVF was plated overnight in condition basal medium for 48 to 72 hours and then harvested. Cells were centrifuged and then resuspended in basal medium eagle (BME; Life Technologies, Grand Island, NY) for 48 to 72 hours (see FIG. 4). ASC-CM was prepared by growing adipose-derived stem cells to confluence before conditioning in BME for 48 hours (see FIG. 4).

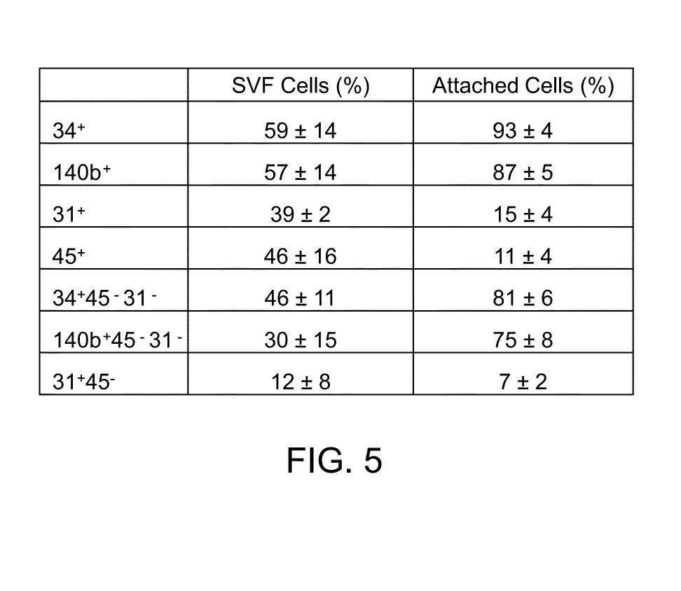

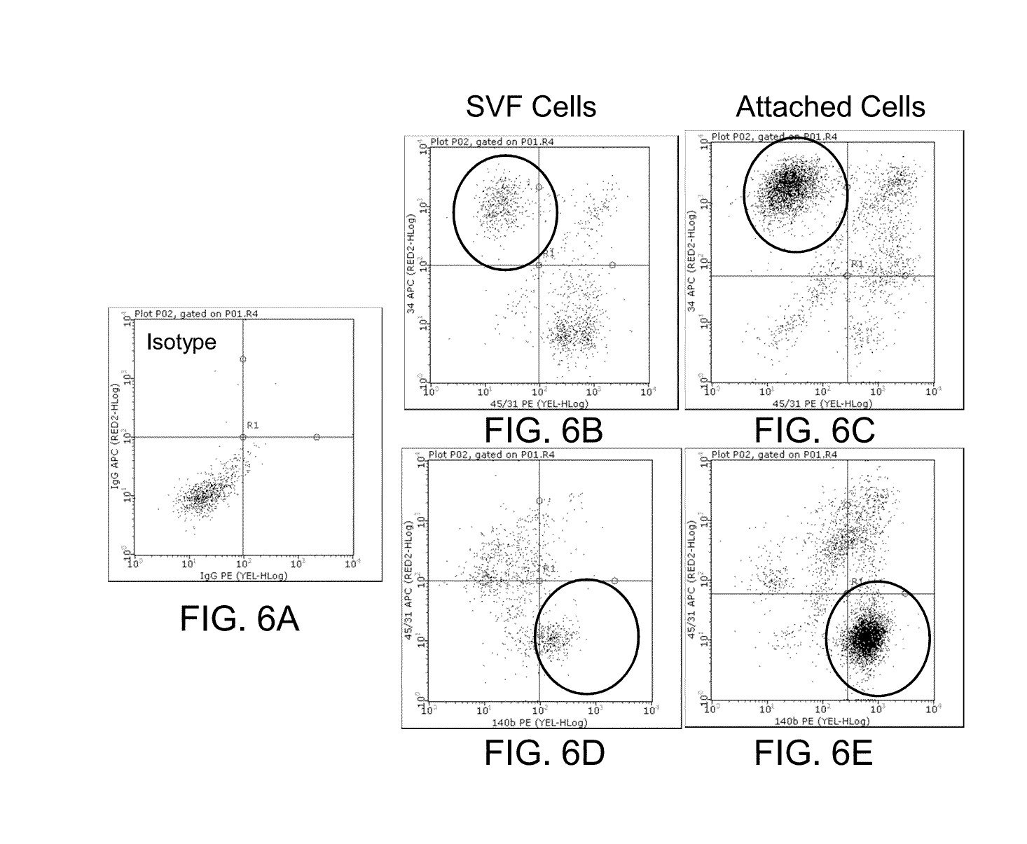

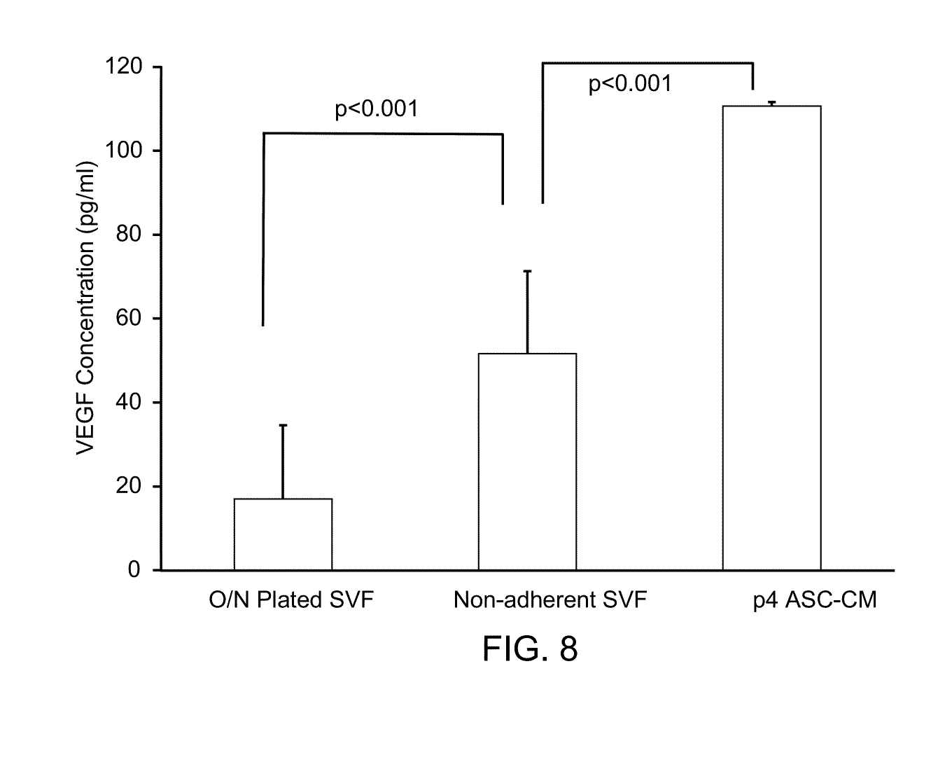

Various characteristics of the SVF-CM and ASC-CM were analyzed. As shown in FIG. 5, expression of various cell-surface molecules was examined to distinguish cell populations obtained by cell separation. Scatter plots of the various cell-surface molecules are shown in FIGS. 6 & 7. Additionally, VEGF concentration in the SVF-CM was analyzed using ELISA and compared to VEGF concentration in ASC-CM. The results are shown in FIG. 8.

### Example 2

In this Example, endothelial cell proliferation assays were performed to analyze cell proliferation in the presence of the SVF-CM as prepared in Example 1 or in the presence of basal condition medium.

Human endothelial cells at passages of 7 or less were seeded at a density of 5000 cells/well in a 48-well plate in EBM-2/5% FBS medium for 24 hours, which limits cell growth. On the following day, the EBM-2/5% FBS medium was then replaced with either basal medium, O/N plated SVF (see FIG. 4), or SVF-CM.

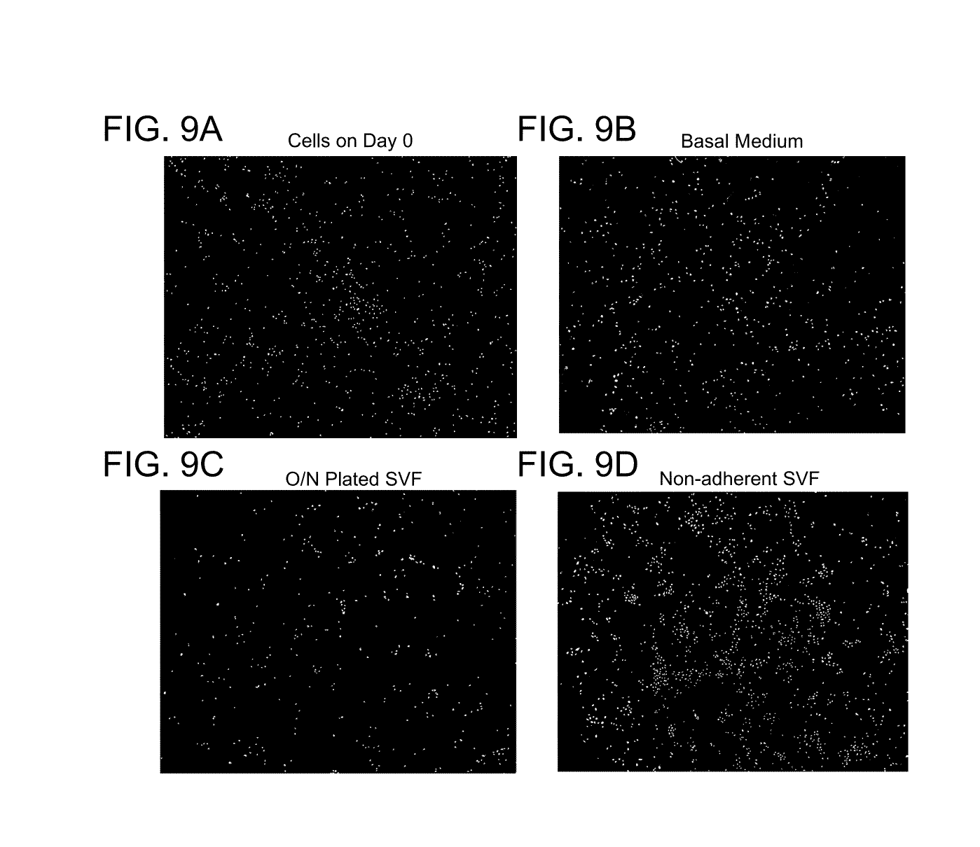

The endothelial cell numbers on day 4 were determined by fixing and staining the cells with DAPI (results shown in FIGS. 9 & 10).

In view of the above, it will be seen that the several advantages of the disclosure are achieved and other advantageous results attained. As various changes could be made in the above description without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. A conditioned medium obtained from digested adipose tissue.

2. The conditioned medium of claim 1, wherein the digested adipose tissue is a fractioned adipose tissue.

3. The conditioned medium of claim 2, wherein the fractioned adipose tissue is selected from the group consisting of adipose-derived stem cells and non-adipocyte, stromal-vascular fraction.

4. A conditioned medium obtained from a non-adipocyte, stromal-vascular fraction.

5. The conditioned medium of claim 4, wherein the conditioned medium is obtained from a suspension culture of the non-adipocyte, stromal-vascular fraction.

6. The conditioned medium of claim 4, wherein the non-adipocyte, stromal-vascular fraction comprises at least one of a leukocyte and an endothelial cell.

7. A method of producing conditioned medium, the method comprising: digesting adipose tissue by contacting the adipose tissue with an enzyme; fractionating the adipose tissue to obtain a fractionated adipose tissue that comprises at least a stromal-vascular fraction; culturing cells obtained from the fractionated tissue in a culture medium; and separating the cells from the culture medium to produce the conditioned medium.

8. The method of claim 7, cells obtained from the stromal-vascular fraction are cultured in the culturing step.

9. The method of claim 8, wherein the stromal-vascular fraction is selected from the group consisting of a freshly isolated population of stromal-vascular fraction and a non-adherent population of stromal-vascular fraction.

10. The method of claim 7, further comprising culturing cells from digested adipose tissue after the digesting step for a sufficient time to allow a plurality of cells to adhere to a culture substrate; collecting non-adherent cells; culturing the non-adherent cells; and collecting culture medium after the culturing of the non-adherent cells, wherein the culture medium results in the conditioned medium.

11. The method of claim 10, wherein the culture substrate comprises a plastic culture dish.

12. The method of claim 11, wherein the plastic culture dish is an uncoated plastic culture dish.

13. The method of claim 10, wherein the culturing the non-adherent cells comprises a suspension culture.

14. The method of claim 13, wherein the conditioned medium is obtained from a non-adipocyte, stromal-vascular fraction of the adipose tissue.
